(19) United States
(12) Patent Application Publication
(10) Pub. No.: US 2012/0116206 A1
(43) Pub. Date: May 10, 2012

(54) Automated non-contrast agent magnetic resonance venography

(75) Inventor: Mitsue Miyazaki, Mount Prospect, IL (US)

(73) Assignees: TOSHIBA MEDICAL SYSTEMS CORPORATION, Otawara-shi (JP); KABUSHIKI KAISHA TOSHIBA, Minato-ku (JP)

(21) Appl. No.: 12/926,270

(22) Filed: Nov. 5, 2010

(51) Int. Cl. A61B 5/055

(52) U.S. Cl. 600/410

(57) ABSTRACT

A magnetic resonance imaging (MRI) system uses an MRI system gantry, an associated controlling computer system and an operator input mechanism, with the controlling computer system including at least one programmed computer configured to effect an automated magnetic resonance venography (MRV) mode. Operator inputs are accepted to preset parameters defining at least one MRV process (i) for acquiring plural sets of image data and (ii) for subsequent interrelated subtractions between the acquired image data sets to produce an MRV image set. The image data sets are thereafter automatically acquired and interrelated subtractions are automatically performed to produce an MRV image data set. The MRV image data set (perhaps after MIP processing) is then output to at least one of (i) an image data memory, (ii) an image display, and (iii) a remote further computer site.

### FIELD

The subject matter below relates generally to magnetic resonance imaging (MRI) processes. The MRI processes described below involve enhancements to magnetic resonance venography (MRV).

### BRIEF DESCRIPTION OF THE DRAWINGS

FIG. 1 is a high-level schematic block diagram of an MRI system adapted to acquire and process MRI data for MRV using an enhanced operator graphical user interface (GUI);

FIG. 2 is a schematic flow chart of exemplary computer program code structure that may be utilized for practicing an exemplary embodiment;

FIG. 3 is a schematic illustration of exemplary SPADE data processing that may be automatically effected by the GUI provided by the program code structure of FIG. 2; and

FIG. 4 is a schematic illustration of an exemplary selective venogram data acquisition, processing and output process.

### DETAILED DESCRIPTION

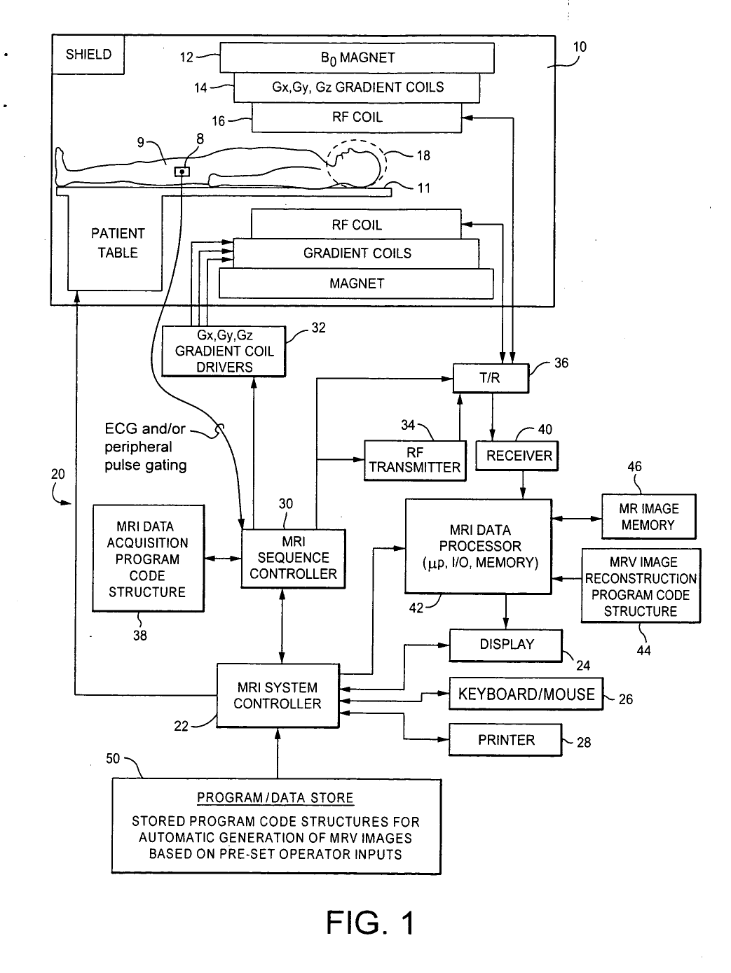

The MRI system shown in FIG. 1 includes a gantry 10 (shown in schematic cross-section) and various related system components 20 interfaced therewith. At least the gantry 10 is typically located in a shielded room. One MRI system geometry depicted in FIG. 1 includes a substantially coaxial cylindrical arrangement of the static field B0 magnet 12, a Gₓ, G_{y} and G_{z} gradient coil set 14 and an RF coil assembly 16. Along the horizontal axis of this cylindrical array of elements is an imaging volume 18 shown as substantially encompassing the head of a patient 9 supported by a patient table 11.

An MRI system controller 22 has input/output ports connected to display 24, keyboard/mouse 26 and printer 28. As will be appreciated, the display 24 may be of the touch-screen variety so that it provides control inputs as well.

The MRI system controller 22 interfaces with MRI sequence controller 30 which, in turn, controls the Gₓ, G_{y} and G_{z} gradient coil drivers 32, as well as the RF transmitter 34 and the transmit/receive switch 36 (if the same RF coil is used for both transmission and reception). Suitable electrode(s) 8 affixed suitably to the patient 9 provide ECG and/or peripheral pulse gating signals to controller 22. The MRI sequence controller 30 includes suitable program code structure 38 for implementing MRI data acquisition sequences already available in the repertoire of the MRI sequence controller 30 to generate diastolic and systolic ECG or peripheral pulse gated images.

The MRI system 20 includes an RF receiver 40 providing input to data processor 42 so as to create processed image data to display 24. The MRI data processor 42 is also configured for access to image reconstruction program code structure 44 and to image memory 46 (e.g., for storing MR image data derived from processing in accordance with the exemplary embodiments and the image reconstruction program code structure 44).

Also illustrated in FIG. 1 is a generalized depiction of an MRI system program/data store 50 where stored program code structures (e.g., for automatic generation of MRV images based on preset operator inputs via a GUI) are stored in computer-readable storage media accessible to the various data processing components of the MRI system. As those in the art will appreciate, the program store 50 may be segmented and directly connected, at least in part, to different ones of the system 20 processing computers having most immediate need for such stored program code structures in their normal operation (i.e., rather than being commonly stored and connected directly to the MRI system controller 22).

Indeed, as those in the art will appreciate, the FIG. 1 depiction is a very high-level simplified diagram of a typical MRI system with some modifications so as to practice exemplary embodiments to be described hereinbelow. The system components can be divided into different logical collections of “boxes” and typically comprise numerous digital signal processors (DSP), microprocessors, special purpose processing circuits (e.g., for fast ND conversions, fast Fourier transforming, array processing, etc.). Each of those processors is typically a clocked “state machine” wherein the physical data processing circuits progress from one physical state to another upon the occurrence of each clock cycle (or predetermined number of clock cycles).

Not only does the physical state of processing circuits (e.g., CPUs, registers, buffers, arithmetic units, etc.) progressively change from one clock cycle to another during the course of operation, the physical states of associated data storage media (e.g., bit storage sites in magnetic storage media) are transformed from one state to another during operation of such a system. For example, at the conclusion of an MR-imaging reconstruction process, an array of computer-readable accessible data value storage sites in physical non-transitory storage media will be transformed from some prior state (e.g., physical state representing all uniform “zero” values or all “one” values) to a new state wherein the physical states at groupings of the physical sites of such an array vary between minimum and maximum digital signal values to represent real world physical events and conditions (e.g., the tissues of a patient over an imaging volume space). As those in the art will appreciate, such arrays of stored digital data values represent and also constitute a physical structure—as does a particular structure of computer control program codes that, when sequentially loaded into instruction registers and executed by one or more CPUs of the MRI system 20, cause a particular sequence of operational states to occur and be transitioned through within the MRI system.

The exemplary embodiments described below provide improved ways to process data acquisitions and/or to generate and display MR-images.

Fresh blood imaging (FBI) techniques can be used in some embodiments described below. FBI is based on an electrocardiogram (ECG) gated or peripheral pulse gated (PPG) three-dimensional (3D) FASE (fast advanced spin echo) technique. It acquires arterial and venous blood flow in a single coronal pass requiring less scan time than other magnetic resonance angiography (MRA) techniques. It also reduces sensitivity to issues like improper ECG timing, turbulent blood flow and differential blood filling that can cause contrast-based MRA to fail. Its main features are: (a) no contrast medium is required and (b) a wide range of 3D data can be acquired in a short time because imaging of the coronal and sagittal planes is possible.

A non-contrast agent magnetic resonance venography (MRV) technique using flow-spoiled (FS) fresh blood imaging (FBI) requires a double image subtraction technique wherein an arterial source or MIP image (which is a diastolic image from which a systolic image has been subtracted) is, in turn, subtracted from another diastolic image.

Especially for the iliac region, the SPADE (single-shot partial dual echo) EPI (echo planar imaging) technique is useful in acquiring three image data sets which allow separation of arteries from veins. For example, three image data acquisitions for implementing SPADE MRV may comprise:
two diastolic SPEED (swap phase encode data) data acquisitions (one with phase encoding (PE) running in the head-feet (HF) direction and one with PE running in the right-left (RL) direction); and
one systolic SPEED data acquisition (with PE running in the RL direction).

In other words, it is well known (e.g., see the SPADE technique) that by acquiring a plurality (e.g., three) image data sets (using suitable systolic and diastolic triggering) and then by suitably nested image subtractions (e.g., of an arterial source image from diastolic HF PE direction images) one can obtain an MRV image of veins.

However, such approaches (e.g., the SPADE technique) have required the operator to separately direct execution of the required multiple image data acquisitions and/or their interrelated and nested subtractions. This is not only cumbersome and error prone but also, because of the elapsed time between image data acquisitions involving separate patient scans, the possibility of unwanted patient anatomy mis-registrations between the various images due to patient movements between scans is undesirably increased.

To address such problems, the exemplary system described below automatically acquires all of the needed image data acquisition sequences substantially without intervening pauses (e.g., three immediately successive image data acquisitions when the operator elects to use the SPADE technique). Then, after the image data acquisition scans are completed, the required subtractions are automatically performed by the system. Image data acquisition sequences that might be pre-selected by the operator for data acquisitions which are then used in various subtraction techniques may, for example, comprise preset operator choices such as:
SPADE (e.g., see above for image acquisition sequences and subtractions)
flow sensitive dephasing (FSD)—FBI
FSD—bSSFP (balanced steady state free precession)
flow rephasing (FR)—FBI
flow dephasing (FD)—FBI
any other sequences that allow separation of arteries from veins by subtraction(s)

For the second through sixth options noted above, the needed data acquisitions may also be selectively made during systolic and/or diastolic ECG intervals.

As explained in more detail below, the exemplary system provides a user selective graphical user interface (GUI) for operator pre-selection of desired MRV procedures (e.g., see above exemplary listing). In this way, all desired image acquisition, subtraction and other related processing such as MIP (maximum intensity projection) processing can be preset by the operator. The system may then automatically proceed to efficiently effect all necessary image data acquisition sequences and subsequent data processing (e.g., subtractions) so as to not only reduce otherwise cumbersome multiple operator-controlled operations, but also to improve the timeliness and quality of the resulting MRV image (e.g., by reducing the probability of patient movement causing mis-registration of patient fluid vessels between image data acquisitions.

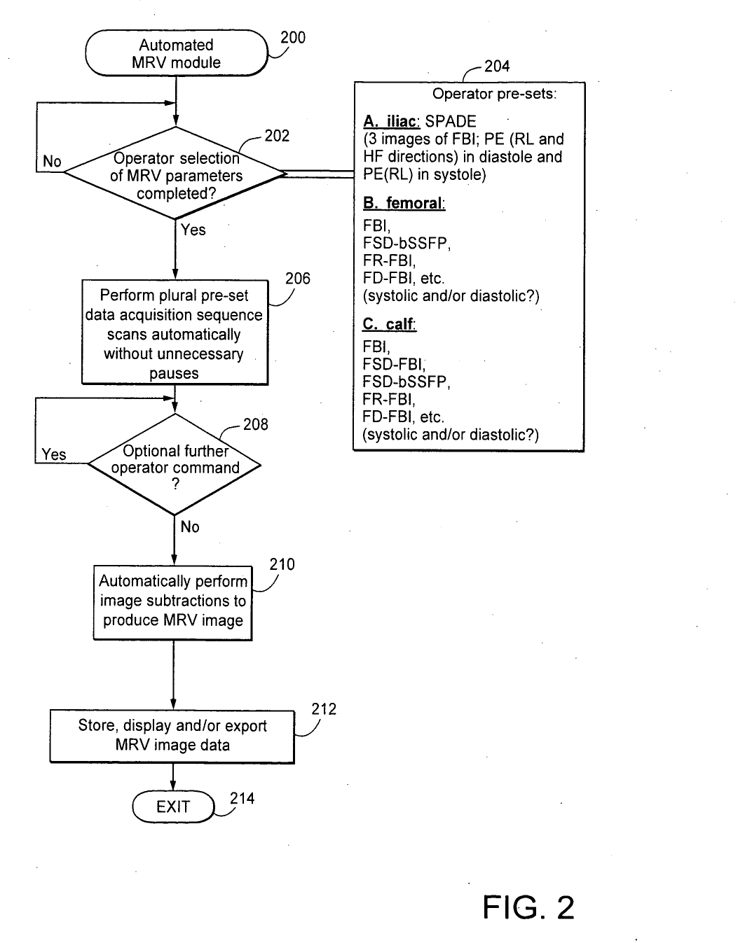

Referring to the exemplary program code structure depicted in FIG. 2, the automated MRV module is entered at 200 (e.g., by suitable operator and/or system transfer of program control).

At data input/wait loop 202, a test is made to see whether operator selection of MRV parameters has been completed. Exemplary operator GUI presets are depicted, for example, at box 204 in FIG. 2. Here, if the iliac region is at issue, then in the preferred exemplary embodiment, the operator will likely chose the SPADE technique described above. On the other hand, if the femoral anatomy is at issue, then the operator may be given other choices as well such as described above and also depicted at box 204 in FIG. 2. Similarly, if the calf anatomy region is at issue, then the same or other MRV parameters may be made available for operator presets. As those in the art will appreciate, this listing of possible operator presets for MRV parameters is merely illustrative and is not intended to be exhaustive or limiting in any respect.

After the preset MRV parameters have been completely entered, loop 202 transfers control to box 206 in FIG. 2. Here, the MRI system proceeds to perform plural preset MRI data acquisition sequence scans automatically and without unnecessary time delays or pauses therebetween (e.g., so as to minimize the risk of patient movement causing blood vessel misalignment between the successively taken images). In essence, the images are taken “simultaneously” insofar as possible in the preferred embodiment.

Although control can pass directly and automatically from box 206 to box 210, it is also possible to interpose an optional operator command at 208 after the plural data acquisition scans have been completed at 206. In any event, box 210 is subsequently entered where the necessary image subtractions are automatically performed to produce the desired MRV image (i.e., in accordance with the operator presets). Thereafter, the MRV image data is stored, displayed and/or exported to some remote system/site or the like at box 212 before exit from this module is taken at 214.

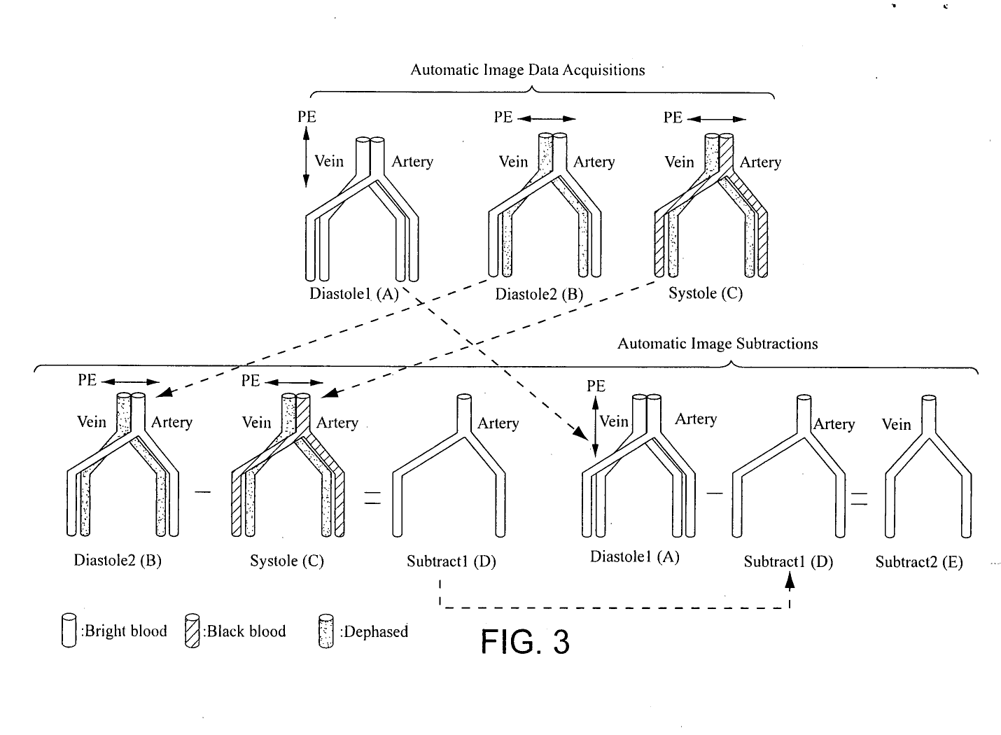

FIG. 3 schematically depicts an exemplary set of data acquisition processes and automatic subtraction processes corresponding to the operator preset for SPADE data processing. As depicted in FIG. 3, the automatic data acquisition first occurs for a Diastole1 (A) image, a Diastole2 (B) image and a Systole (C) image. The Systole (C) image is then subtracted from the Diastole2 (B) image to produce an arterial image that is subsequently then subtracted from the Diastole1 (A) image to produce the desired Subtract2 (E) output MRV image data set.

Data acquisition for the SPADE technique (e.g., as preferred for the iliac region) involves three (3) data set acquisition scans: PE (RL and HF directions) during diastole and PE (RL) during systole. Data acquisition for other areas (e.g., for the femoral and calf regions) may involve: FBI, FSD-FBI, FSD-bSSFP, FR-FBI, or FD-FBI, etc possible selections (with further selections possible for using only systolic images and/or using both systolic and diastolic image data acquisitions).

Data acquisitions for the SPADE technique involves: subtracted image (A) of systolic (PE=RL) from diastolic (PE=RL) images and further subtraction of the subtracted image (A) from the diastolic (PE=HF), followed by MIP (maximum image projection) processing.

For other MRV techniques, such as FBI, FD-FBI or FSD-FBI, etc. the processing may involve: subtraction for both systolic and diastolic acquisitions, followed by MIP (of if only systolic images are used, only MIP processing may be required or desired).

In the preferred exemplary embodiment, a GUI for MRV is provided, wherein:
1. The system acquires all required image data acquisition sequences “simultaneously” (i.e., without unnecessary delays or pauses when acquiring image data for the SPADE selection).
2. After the image data acquisitions, all required subtractions are automatically performed by the system in accordance with operator pre-selections.
3. The image sequences involved could be FD-FBI, Flow-sensitive dephasing (FSD)-FBI, FSD-bSSFP, and/or any other sequences that allow separation of arteries from veins in output MRV images.

The exemplary user selective GUI permits operator selection of SPADE, FS-FBI, FSD-FBI and FSD-bSSFP, etc. Thereafter, all subtraction and/or MIP processing steps are preset and automatically processed.

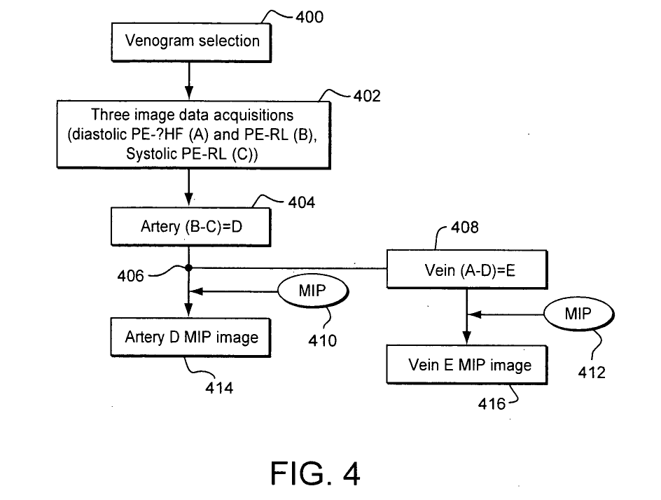

An alternate embodiment is depicted at FIG. 4 were venogram selection can result in output images of either arteries or veins. After the routine is entered at box 400 (e.g., with an operator input selection for output artery images or vein images), then the three SPADE image data acquisitions are executed at 402. Then at box 404, an image subtraction process (B-C) produces an arterial image D. As depicted in FIG. 4, depending upon the venogram operator selection at 400, a branch may be made at 406 to box 408 where a further image subtraction process is effected (A-D) so as to produce an image E of veins. Of course, as will be understood by those in the art, the venogram selection at 400 may include a request by the operator to output images of both arteries and veins.

In any event, MIP processing is performed at 410 and 412 to produce final output artery and vein MIP images D and E, respectively, at 414 and 416. As with the earlier embodiments, these final output images may be output to display, to non-transitory digital storage media or exported outside the originating MRI system.

While certain embodiments of the inventions have been described, these embodiments have been presented by way of example only, and are not intended to limit the scope of the inventions. Indeed, the novel methods and systems described herein may be embodied in a variety of other forms. Furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A magnetic resonance imaging (MRI) system comprising:
an MRI system gantry, an associated controlling computer system and an operator input mechanism, said controlling computer system including at least one programmed computer configured to effect an automated magnetic resonance venography (MRV) mode wherein:
(a) operator inputs are accepted to preset parameters defining at least one MRV process (i) for acquiring plural sets of image data and (ii) for subsequent interrelated subtractions between said acquired image data sets to produce an MRV image set;
(b) said sets of image data are thereafter automatically acquired;
(c) said interrelated subtractions are thereafter automatically effected to produce said MRV image data set; and
(d) said MRV image data set is output to at least one of (i) an image data memory, (ii) an image display, and (iii) a remote further computer site.

2. An MRI system as in claim 1, wherein maximum intensity projection (MIP) processing of said MRV image data sets is performed prior to being output.

3. An MRI system as in claim 1, wherein all of said operator inputs are input prior to image data acquisition.

4. An MRI system as in claim 1, wherein a further operator input is required before said interrelated subtractions are effected.

5. An MRI system as in claim 1, wherein said acquired image data sets are acquired substantially without time delay between immediately successive data acquisition sequences.

6. An MRI system as in claim 1, wherein said operator inputs comprise selecting one of the following MRV scan sequence options:
(i) SPADE (single shot partial dual echo) EPI (echo planar imaging),
(ii) fresh blood imaging (FBI),
(iii) flow sensitive dephasing (FSD)—FBI,
(iv) FSD—balanced steady state free precession (bSSFP),
(v) flow rephasing (FR)—FBI, and
(vi) flow dephasing (FD)—FBI.

7. A magnetic resonance imaging (MRI) method comprising:
using an MRI system gantry, an associated controlling computer system and an operator input mechanism to effect an automated magnetic resonance venography (MRV) mode wherein:
(a) operator inputs preset parameters defining at least one MRV process (i) for acquiring plural sets of image data and (ii) for subsequent interrelated subtractions between said acquired image data sets to produce an MRV image set;
(b) said sets of image data are thereafter automatically acquired;
(c) said interrelated subtractions are thereafter automatically effected to produce said MRV image data set; and
(d) said MRV image data set is output to at least one of (i) an image data memory, (ii) an image display and (iii) a remote further computer site.

8. An MRI system as in claim 7, wherein step (c) also includes maximum intensity projection (MIP) processing of image data sets.

9. An MRI system as in claim 7, wherein all of said operator inputs are input prior to image data acquisition.

10. An MRI system as in claim 7, wherein a further operator input is required before said interrelated subtractions are effected.

11. An MRI system as in claim 7, wherein said acquired image data sets are acquired substantially without time delay between immediately successive data acquisition sequences.

12. An MRI system as in claim 7, wherein said operator inputs comprise selecting one of the following MRV scan sequence options:
(i) SPADE (single shot partial dual echo) EPI (echo planar imaging),
(ii) fresh blood imaging (FBI),
(iii) flow sensitive dephasing (FSD)—FBI,
(iv) FSD—balanced steady state free precession (bSSFP),
(v) flow rephasing (FR)—FBI, and
(vi) flow dephasing (FD)—FBI.

13. A non-transitory computer program storage medium containing executable computer program code for controlling an MRI system including at least one programmed computer configured to effect an automated magnetic resonance venography (MRV) mode when said executable computer program code is executed, and wherein:
(a) operator inputs preset parameters defining at least one MRV process (i) for acquiring plural sets of image data and (ii) for subsequent interrelated subtractions between said acquired image data sets to produce an MRV image set;
(b) said sets of image data are thereafter automatically acquired;
(c) said interrelated subtractions are thereafter automatically effected to produce said MRV image data set; and
(d) said MRV image data set is output to at least one of (i) an image data memory, (ii) an image display, and (iii) a remote further computer site.

14. A non-transitory computer program storage medium as in claim 13, wherein step (c) also includes maximum intensity projection (MIP) processing of image data sets.

15. A non-transitory computer program storage medium as in claim 13, wherein all of said operator inputs are input prior to image data acquisition.

16. A non-transitory computer program storage medium as in claim 13, wherein a further operator input is required before said interrelated subtractions are effected.

17. A non-transitory computer program storage medium as in claim 13, wherein said acquired image data sets are acquired substantially without time delay between immediately successive data acquisition sequences.

18. A non-transitory computer program storage medium as in claim 13, wherein said operator inputs comprise selecting one of the following MRV scan sequence options:
(i) SPADE (single shot partial dual echo) EPI (echo planar imaging),
(ii) fresh blood imaging (FBI),
(iii) flow sensitive dephasing (FSD)—FBI,
(iv) FSD—balanced steady state free precession (bSSFP),
(v) flow rephasing (FR)—FBI, and
(vi) flow dephasing (FD)—FBI.
